(19) United States
(12) Patent Application Publication
(10) Pub. No.: US 2016/0271026 A1
(43) Pub. Date: Sep. 22, 2016

(54) GEMSTONE INFUSED LIFESTYLE PRODUCTS

(71) Applicant: Catherine B. E. Cooper, Marina Del Rey, CA (US)

(72) Inventor: Catherine B. E. Cooper, Marina Del Rey, CA (US)

(21) Appl. No.: 15/073,314

(22) Filed: Mar. 17, 2016

Related U.S. Application Data
(60) Provisional application No. 62/136,380, filed on Mar. 20, 2015.

(51) Int. Cl. A61K 8/19; A61Q 1/04; A61K 8/25; A61K 8/26; A45D 34/02; A61Q 13/00; A61K 8/02; A61K 8/27; A61K 8/23; C11B 9/00; A61Q 3/02; A61K 8/28

(52) U.S. Cl. CPC A61K 8/19; A61Q 3/02; A61Q 1/04; A61K 8/25; A61K 8/26; A61K 8/28; A61Q 13/00; A61K 8/0216; A61K 8/27; A61K 8/23; C11B 9/00; A45D 34/02; A61K 2800/5922; A61K 2800/262

(57) ABSTRACT

Gemstone infused product to enhance beauty, evoke emotion and amplify essence of a user is disclosed. In accordance with one embodiment of the present disclosure, the gemstone infused product comprises a container containing a fluid selected from perfume/oil, fragrant, nail polish, water or lip gloss; a plurality of gemstones or combination of gemstones and simulants are placed inside the transparent container and covered by the fluid; wherein the gemstones and simulants are not pulverized, emulsified or powdered. The infusion of gemstone enhances physiological, emotional and/or metaphysical properties of the surrounding environment. The combination of different gemstones and crystals offer the user with an ability to select types of enhancement desired by the user and may be used in household decor and or jewelry.

### CROSS-REFERENCE TO RELATED PATENT APPLICATIONS

Embodiments of the present invention relate to U.S. Provisional Application Ser. No. 62/136,380, filed Mar. 30, 2015, entitled “Gemstone Infused Lifestyle Products”, the content of which is incorporated by reference herein in its entirety and which is basis for a claim of priority.

### FIELD OF THE DISCLOSURE

The present disclosure generally relates to gemstone infused lifestyle product. Living with gems is a lifestyle enhancement concept. More specifically, the present disclosure discloses gemstone infused lifestyle product that has the capability to enhance beauty, evoke emotion and amplify essence through metaphysical properties of a user through the combination of loose rough, polished, faceted, natural, and synthetic gemstone, crystal and/or minerals.

### BACKGROUND OF THE INVENTION

Although it is well known in the art to use crushed or powdered minerals and/or gemstones in beauty and cosmetic products, the products in the conventional art typically require the minerals and/or gemstones to be emulsified and powdered within the product. The emulsified and/or powdered minerals and/or gemstones are limited to products that can be safely worn and possibly absorbed into the skin of a user. This obliteration of the minerals and/or gemstones does not necessarily provide a user with an advantageous product capable of harnessing the essence of various minerals or gemstones in personal use products. Additionally, by emulsifying and powdering the minerals and/or gemstones and placing the pulverized minerals and/or gemstones directly in ointments, creams or elixirs, existing art is limited to the biocompatibilities of the mineral and/or gemstones themselves and additionally in relation to the other product ingredients.

Accordingly, there exists a need for improved infusion of gemstone and/or minerals in lifestyle enhancement products that enhances beauty, evokes emotion and amplifies essence of a user through combination of loose rough, polished, natural, synthetic, and faceted gemstone, crystal and/or minerals.

### SUMMARY OF THE INVENTION

The present disclosure generally relates to gemstone infused lifestyle product. More specifically, the present disclosure discloses gemstone infused lifestyle product that enhances beauty, evokes emotion and amplifies essence through metaphysical properties of a user through the combination of loose rough, polished, and faceted gemstone, crystal and/or minerals.

In accordance with one aspect of at least one embodiment of the present disclosure, a gemstone infused product is disclosed. The product comprises: 1) a container containing a fluid selected from perfume oil, fragrant water, nail polish or lip gloss; 2) a plurality of gemstones or combination of gemstones and simulants are placed inside the transparent container and covered by the fluid. The gemstones and simulants are not pulverized, emulsified or powdered. The infusion of gemstone has the potential to enhance physiological, emotional and/or metaphysical properties of the surrounding environment. The combination of different gemstones and crystals offer the user with an ability to select types of enhancement desired by the user and most of the gemstones used are reusable.

The embodiments of the present invention allows a variety of combination of compatible and customized pairing/coordination of various gemstones and mineral properties to maximize the ability of the products to affect various targeted aspects to increase physiological and metaphysical properties to enhance beauty, evoke emotions, and amplify the essence of a user's presence while providing a unique offering not yet available in the marketplace.

These and other features, aspects and advantages of the present invention will become better understood with reference to the following description and claims.

### DETAILED DESCRIPTION OF THE PREFERRED EMBODIMENT(S) AND INVENTION

The following description is not to be taken in a limiting sense, but is made merely for the purpose of illustrating the general principles of the disclosure, since the scope of the disclosure is best defined by the following claims. Various inventive features are described below that can each be used independently of one another or in combination with other features.

For purposes of the present disclosure, the term “gemstone” or “gem” generally refers to a piece of mineral crystal, natural or synthetic, that can be used to make jewelry or other adornments. In addition, certain rocks (such as Lapis Lazuli) or organic materials that are not minerals (such as Amber, Coral, Pearl or Jet) are also considered to be gemstones within the scope of present disclosure. For example and not by way of limitation, the gemstone families of present disclosure include Amethyst, Apatite, Beryl (Aquamarine, Bivite, Emerald, Goshenite, Heiodor, Morganite), Calcedony/Agate, Chrysoberyl, Citrine, Corundum, Diamond, Diopside, Feldspar, Fluorite, Garnet, Glass Tekites, Hematite, Hemimorphite, Iolite, Jade, Kyanite, Lapis Lazuli, Malachite, Opal, Peridot, Prehite, Quarts, Rhodochrisite, Scapolite, Sillimanite, Sphene, Spinel, Spodumene, Sugilite, Topaz, Tourmaline, Tugtupite, Turquoise, Zircon, and Zoisite.

In addition, natural and/or synthetic gemstone combined with simulants will be the combination for all products mentioned throughout the specification. So the combinations include but are not limited to at least the following: 1) Natural Gemstones only, 2) Synthetic gemstones only, 3) Natural Gemstones and Simulants, 4) Synthetic Gemstones and Simulants, 5) Natural and Synthetic Gemstones and 6) Combination of natural and synthetic gemstones with simulants added to enhance sparkle. A natural gemstone should have the same chemical composition, physical and optical properties as the synthetic gemstone. It is usually the rarity and value that set them apart.

Broadly, embodiments of the present disclosure generally provide a gemstone infused product, the product comprises: a container containing a fluid that is selected from water, perfume/oil, fragrant water, nail polish or lip gloss; a plurality of gemstones or combination of both gemstones and simulants are placed inside the transparent container and covered by the fluid. The selected gemstones and simulants are not pulverized, emulsified or powdered.

The gemstone families may include, but not limited to, Amethyst, Apatite, Beryl (Aquamarine, Bivite, Emerald, Goshenite, Heiodor, Morganite), Calcedony/Agate, Chrysoberyl, Citrine, Corundum, Diamond, Diopside, Feldspar, Fluorite, Garnet, Glass Tekites, Hematite, Hemimorphite, Iolite, Jade, Kyanite, Lapis Lazuli, Malachite, Opal, Peridot, Prehite, Quarts, Rhodochrisite, Scapolite, Sillimanite, Sphene, Spinel, Spodumene, Sugilite, Topaz, Tourmaline, Tugtupite, Turquoise, Zircon, and Zoisite. The simulants may include Yttrium Aluminium Garnet (YAG), Gadolinium gallium garnet (GGG), Cubic Zirconia and Glass Zircon.

The gemstones and/or simulants can optionally be loose rough, polished, and faceted. Example of faceted cut may include, but not limited to, Round, Brilliant, Briolette, Mixed, Fancy, Step, Square, Cross, French, Emerald, Asscher, Barrell, Antique, Ball, Needle, Old Mine, Rose, Star, Pear, Heart, Oval, Out of Round, Pentagon, Olive, Ceylon, Magna, Petal, Tycoon, Octagon, Triangle, Baguette, Radiant, Princess or combination thereof.

Table 1 below shows an example of Round MM sizes contemplated in the Present invention. Charts for each cut/carat vary per stone

TABLE 1

| | Round-“mm” | | | | | | | | | | |
|---|---|---|---|---|---|---|---|---|---|---|---|
| | 2 | 2.5 | 3 | 3.5 | 4 | 5 | 6 | 7 | 8 | 9 | 10 |
| Amethyst/Citrine | 0.03 | 0.05 | 0.1 | 0.15 | 0.24 | 0.4 | 0.7 | 1.1 | 1.8 | 2.55 | 3.3 |
| Aquamarine | 0.03 | 0.05 | 0.08 | 0.15 | 0.22 | 0.4 | 0.42 | 1.2 | 2 | 2.75 | 3.5 |
| Emerald | 0.03 | 0.06 | 0.1 | 0.17 | 0.26 | 0.52 | 0.95 | 1.5 | 2.2 | 2.95 | 3.75 |
| Diamond | 0.04 | 0.07 | 0.1 | 0.17 | 0.23 | 0.5 | 0.9 | 1.4 | 1.9 | 3.05 | 4.25 |
| Garnet | 0.04 | 0.07 | 0.11 | 0.23 | 0.31 | 0.56 | 1 | 1.6 | 2.2 | 2.75 | 4.15 |
| Opal | 0.02 | 0.04 | 0.06 | 0.11 | 0.16 | 0.34 | 0.57 | 0.83 | 1.4 | 1.95 | 2.55 |
| Peridot | 0.03 | 0.05 | 0.11 | 0.15 | 0.3 | 0.53 | 0.85 | 1.2 | 2 | 2.39 | 4.38 |
| Ruby/Sapphire | 0.05 | 0.09 | 0.14 | 0.22 | 0.38 | 0.65 | 1.2 | 1.8 | 2.4 | 3.45 | 4.5 |
| Topaz | 0.04 | 0.09 | 0.11 | 0.2 | 0.34 | 0.56 | 1 | 1.5 | 2 | 3.3 | 4.4 |
| Tourmaline | 0.04 | 0.08 | 0.12 | 0.17 | 0.25 | 0.45 | 0.8 | 1.3 | 2 | 2.6 | 3.35 |
| Zircon | 0.06 | 0.1 | 0.16 | 0.26 | 0.4 | | | | | | |

Gemstones, minerals and crystals have rarity, durability and offer profound beauty. They contain essences through metaphysical properties. These properties have historically been useful in evoking feelings and providing spiritual healing. The disclosed product combines all of these properties, which increasing beauty and allowing user to live with gems as a common day lifestyle practice through cosmetics, home decor and jewelry.

In one embodiment of the present disclosure, when the fluid in the gemstone infused product is lip gloss, the container of the product can further include a cap enclosing the container. Preferably, the container is transparent or light color to show off the gemstone, minerals, and crystals inside. The container may or may not be colored. Gemstones, minerals, and crystals can be removed once the lip gloss fluid is finished as a token for the user to continue to use in home decor products and/or jewelry items. Home decor products include but not limited to: Lamps/Fillable Lamps/Shades, Chandeliers, Mobiles, Wall and Stand Alone Mirrors, Wall and Picture Frames, Votive Holders, Vases, Napkin Rings, Placecard Holders, Wall Art and Dream Catchers, Wall and Table Clocks, Utensils, Salt & Pepper Shakers, Curtain Rod Bulbs, Drawer and Cabinet Pulls/Knobs, Reed Diffusers/Room Spray, Room Mist, Oil Bulbs. Jewelry items include but are not limited to: Necklaces, Earrings, Pendants, Lockets, Brooches, Rings, Bracelets, Cuff Links, Armlets, Charms, Barrettes, Watch, Belt buckle.

In yet another embodiment of the present disclosure, the gemstone infused product does not contain any fluid and simply contains a plurality of gemstones or a combination of both gemstones and simulants which are placed inside a container, preferably transparent container. The selected gemstones and simulants are not pulverized, emulsified or powdered.

In yet another embodiment of the present disclosure, the gemstone infused product does not contain any fluid and containers. The gemstone infused products can be anything that can contain a plurality of gemstones or combination of gemstones and simulants such as the home decor products and/or jewelry items described above. The selected gemstones and/or simulants are not pulverized, emulsified or powdered. The disclosed gemstone infused product has the capability to enhance beauty, evoke emotion and amplify essence through metaphysical properties of a user through different combination of gemstones and/or simulants.

Below are examples of combination of gemstones that can be used for lip gloss.

### COSMETICS—SPARKLE STYLE TYPE GEM INFUSED LIP GLOSS

A. Types of Gemstones/Crystals to Be Infused with Lip Gloss and Cut:

Loose gemstones, minerals and crystals that are Rare, Precious, Semi-Precious, and possible synthetic of various cut, color, saturation, shape, Mohs Hardness, Specific Gravity and possible treatment can be used.

Gemstone/Crystal size of polished/faceted may include, but are not limited to following Gem Species and variety family of stones: Amethyst, Apatite, Beryl (Aquamarine, Bivite, Emerald, Goshenite, Heiodor, Morganite), Calcedony/Agate, Chrysoberyl, Citrine, Corundum, Diamond, Diopside, Feldspar, Fluorite, Garnet, Glass Tekites, Hematite, Hemimorphite, Iolite, Jade, Kyanite, Lapis Lazuli, Malachite, Opal, Peridot, Prehite, Quarts, Rhodochrisite, Scapolite, Sillimanite, Sphene, Spinel, Spodumene, Sugilite, Topaz, Tourmaline, Tugtupite, Turquoise, Zircon, and Zoisite.

Embodied cut examples may include, but are not limited to or meant to exclude others not listed: Round, Brilliant, Briolette, Mixed, Fancy, Step, Square, Cross, French, Emerald, Asscher, Barrell, Antique, Ball, Needle, Old Mine, Rose, Star, Pear, Heart, Oval, Out of Round, Pentagon, Olive, Ceylon, Magna, Petal, Tycoon, Octagon, Triangle, Baguette, Radiant, Princess.

The embodied Gemstone/Crystal size of rough may include, but are not limited to or meant to exclude others not listed in these species, variety, or families of Crystal Habits and Crystal Systems: Cubic, Tetragonal, Hexagonal, Trigonal, Orthorhombic, Monoclinic, Triclinic. The gemstone infused lip gloss may also include gemstones with inclusions, pleochroism, and optical phenomena; such as star stones and cat's-eyes.

Man made simulants may be added to any formulation for additional sparkle factor and may include YAG, GGG, Cubic Zirconia, Glass Zircon. Sparkle Style type applications may determine gemstone, mineral combinations based on metaphysical properties and desired essences. Birthstones may also be incorporated.
B. Example Combinations for Gemstone Infused Lip Gloss:
   1) Clear Lip Gloss containing Amethyst, Blue Topaz, and Cubic Zirconia. This is believed to provide strength and emotional balance, the stone of change merged with the stone to attain clarity and manifest desires.
   2) Clear Lip Gloss containing Peridot, Citrine and Cubic Zirconia. This is believed to emotes abundance and success with emotional balance and protection of greed.
   3) Clear Lip Gloss Containing Rose Quartz, Garnet and Cubic Zirconia. This is believed to attracts the energy of love and sensuality. Stone of love merged with stone of health.
   4) Clear Lip Gloss Containing Amethyst, Blue Topaz, Peridot, Citrine, Garnet, and Cubic Zirconia. It is believed that this gemstone extravaganza will provide a balance of love, health, clarity, strength, and success.

All of these gemstone infusion combinations above may be additionally coupled with any the following exemplary offerings: clear lip gloss formula with mica for additional sparkle clear lip gloss lip plumping formula, clear lip gloss lip plumping formula with mica for additional sparkle, colored pink transparent tints, colored pink transparent tints with mica for additional sparkle, and/or colored pink transparent tints with lip plumping.

In yet another embodiment of the present disclosure, the combination of gemstones infused in the lip gloss is based on birthday/Birthstone referring to Table 2 below:

TABLE 2
Sparkle Style Gemstone Infusion Birthstone List for Lip
Gloss, Perfume Oils, Mist, Nail Polish

| Month | Gemstone | Purported Effect |
|---|---|---|
| January | Garnet | Stone of Health |
| February | Amethyst | Peace and Inner Strength |
| March | Aquamarine | Calm and Clarity |
| April | Diamond | Stone of Light |
| May | Emerald | Love and Truth |
| June | Pearl | Purity and Truth |
| July | Ruby | Happiness |
| August | Peridot | Abundance and Prosperity |
| September | Sapphire | Creative Expression |
| October | Opal | Luck and Offers Protection |
| November | Citrine | Success |
| December | Topaz | Emotional Balance |

All birthstone lip gloss may optionally include: clear lip gloss, clear lip gloss formula with mica for additional sparkle, clear lip gloss lip plumping formula, possible transparent color tints.

In yet another embodiment of the present disclosure, when the fluid in the gemstone infused product is fragrant water or perfume oil, the container of the product may further include a decorative item that floats on the fragrant water or perfume oil such as a candle or a flower. Preferably, the container is transparent or light color so what is inside the container can be clearly seen. Below are examples of combination of gemstones that can be used for fragrant water or perfume oil.

### COSMETICS—SPARKLE STYLE GEMSTONE INFUSED PERFUME OIL

A. Types of Gemstones/Crystals to Be Infused with Lip Gloss and Cut:

Loose Gemstone, Mineral, and Crystals contemplated in the present invention include, but are not limited to: Rare, Precious, Semi-Precious, and possible synthetic of various cut, color, saturation, shape, Mohs Hardness, Specific Gravity and possible treatment.

Embodied Gemstone, Mineral, and Crystal size of rough, loose, polished or faceted will include, but are not limited to these Species and Variety of gemstones/minerals: Apatite, Beryl (Aquamarine, Bivite, Emerald, Goshenite, Heiodor, Morganite), Calcedony/Agate, Chrysoberyl, Corundum, Diamond, bDiopside, Feldspar, Fluorite, Garnet, Glass Tekites, Hematite, Hemimorphite, Iolite, Jade, Kyanite, Lapis Lazuli, Malachite, Opal, Crystal Systems: Cubic, Tetragonal, Hexagonal, Trigonal, Orthorhombic, Monoclinic, Triclinic May include gemstones with inclusions, pleochroism, and optical phenomena; such as star stones and cat's-eyes.

Man made simulants may be added to any formulation for additional sparkle factor and may include YAG, GGG, Cubic Zirconia, Glass Zircon as well as additional simulants.
B. Example Combinations for Gemstone Infused Perfume/Oil:
   1) Transparent Perfume/Oil, water containing Amethyst, Blue Topaz, and Cubic Zirconia. This is believed to provide strength and emotional balance, the stone of change merged with the stone to attain clarity and manifest desires.
   2) Transparent Perfume/Oil, water containing Peridot, Citrine and Cubic Zirconia. This is believed to emote abundance and success with emotional balance and protection of greed.
   3) Transparent Perfume/Oil, water Containing Rose Quartz, Garnet and Cubic Zirconia. This is believed to attract the energy of love and sensuality, where stone of love is merged with stone of health.
   4) Transparent Perfume/Oil, water containing Amethyst, Blue Topaz, Peridot, Citrine, Garnet, and Cubic Zirconia. This gemstone extravaganza is believed to provide a balance of love, health, clarity, strength, and success.

In yet another embodiment of the present disclosure, the combination of gemstones infused in the Perfume/oil is based on birthday/Birthstones referring to Table 2.

### COSMETICS—SPARKLE STYLE TYPE GEM INFUSED NAIL POLISH

A. Types of Gemstones/Crystals to Be Infused with Nail Polish and Cut:

Loose Gemstone, Mineral, and Crystals contemplated in the present invention include, but are not limited to: Rare, Precious, Semi-Precious, and possible synthetic of various cut, color, saturation, shape, Mohs Hardness, Specific Gravity and possible treatment.

Embodied Gemstone, Mineral, and Crystal size of rough, loose, polished or faceted will include, but are not limited to these Species and Variety of gemstones/minerals: Apatite, Beryl (Aquamarine, Bivite, Emerald, Goshenite, Heiodor, Morganite), Calcedony/Agate, Chrysoberyl, Corundum, Diamond, bDiopside, Feldspar, Fluorite, Garnet, Glass Tekites, Hematite, Hemimorphite, Iolite, Jade, Kyanite, Lapis Lazuli, Malachite, Opal, Crystal Systems: Cubic, Tetragonal, Hexagonal, Trigonal, Orthorhombic, Monoclinic, Triclinic May include gemstones with inclusions, pleochroism, and optical phenomena; such as star stones and cat's-eyes.

Man made simulants may be added to any formulation for additional sparkle factor and may include YAG, GGG, Cubic Zirconia, Glass Zircon as well as additional simulants
B. Example Combinations for Gemstone Infused Nail Polish:
   1) Transparent Nail Polish containing Amethyst, Blue Topaz, and Cubic Zirconia. This is believed to provide strength and emotional balance, the stone of change merged with the stone to attain clarity and manifest desires.
   2) Transparent Nail Polish containing Peridot, Citrine and Cubic Zirconia. This is believed to emote abundance and success with emotional balance and protection of greed.
   3) Transparent Nail Polish containing Rose Quartz, Garnet and Cubic Zirconia. This is believed to attract the energy of love and sensuality, where stone of love is merged with stone of health.
   4) Transparent Nail Polish containing Amethyst, Blue Topaz, Peridot, Citrine, Garnet, and Cubic Zirconia. This gemstone extravaganza is believed to provide a balance of love, health, clarity, strength, and success.

In yet another embodiment of the present disclosure, the combination of gemstones infused in the Nail Polish is based on birthday/Birthstones referring to Table 2.

In addition, following non-exclusive list of minerals or gemstones may also be used in the gemstone infused product disclosed by the present disclosure. Their purported physiological, metaphysical or general physiological effects are shown in Table 3 below. A user may combine different types of gemstones and/or crystals depending on the user's desire or requirement.

TABLE 3

| | Purported Physiological, Metaphysical or General |
|---|---|
| Gemstone | Psychological Effect |
| Agate | Balances ying-yang energy, stabilizes the aura. |
| | Facilitates discernment. Imparts strength and |
| | courage. Opens one to innate creative talents. (There |
| | are many forms of agate, each with specific |
| | properties.) |
| Amber | Purifies body, mind, and spirit. Balances |
| | electromagnetics of the body and allows even flow of |
| | energies. Provides a positive, soothing energy. |
| | Spiritualizes the intellect. |
| Amethyst | Sedative energy. Facilitates spirituality and |
| | contentment. Stone of stability, strength, and peace. |
| | Excellent for meditation. Enhances psychic ability. |
| Aventurine | Independence, leadership, creativity. Balances |
| | male/female energies. Aligns mental, physical, |
| | emotional, and auric bodies. |
| Aquamarine | Courage, intellect, protection. Assists spiritual |
| | awareness, actualization. |
| Azurite | Awakens psychic ability, insight, intuition. Third |
| | eye. Excellent for clearing the mind in meditation. |
| Apache Tears | Assists in the grieving process, and allows |
| | forgiveness. Breaks down the barriers that hold one |
| | back. Also good for the analytical mind. |
| Amazonite | Balancing energy, harmony, universal love. |
| Calcite | Energy amplifier, teacher. Optical Calcite is excellent |
| | for clearing and cleansing. |
| Celestite | Excellent stone for dream recall and astral travel. |
| | Hope and harmony. Communication. |
| Chrysocolla | Strength, balance. Promotes harmony and |
| | attunement to the earth. Purifies one's environment. |
| Chrysoprase | Balances yin-yang energy. Acceptance of self and |
| | others. |
| Citrine | Positive energy - stone needs no cleansing. |
| | Dissipates negative energy. Warmth, joy, optimism. |
| Copal | Excellent for strengthening the abilities of the mind, |
| | and altered states of consciousness. Amplifies one's |
| | energy field. |
| Diamond | Purity, perfection, abundance, inspiration. |
| Emerald | Loyalty, sensitivity, harmony, tranquility. Assists in |
| | memory retention and mental clarity. |
| Fluorite | Stability, order, discernment, concentration. Helps |
| | one to understand and maintain ideals and the |
| | perfection of the universe. |
| Garnet | Commitment, devotion, love, stability and order. |
| | Even flow of energy. |
| Gold | Spirituality, understanding, attunement to nature. |
| | Attracts positive energy. Excellent healer. |
| Hematite | Excellent for the mind. Grounding. |
| Herkimer | Awareness, attunement, clairvoyance, telepathy. |
| Diamond | Good for information retention. |
| Jade | Harmony, peace, fidelity, confidence. A wonderful |
| | dream stone. |
| Jasper | Protection, awareness, insight. Grounding. |
| Kunzite | Communication, love, peace. Protects and dissolves |
| | negativity. Excellent for meditation. |
| Kyanite | Never needs cleaning or clearing, aligns all chakras. |
| | Tranquility, communication, psychic awareness. |
| | Excellent for meditation and dream recall. |
| Labradorite | Represents the light of the universe, extra-terrestrial |
| | energy. Intuition, subconscious. Illumination. |
| Lapis Lazuli | Knowledge, wisdom, perfection, protection, creative |
| | expression. |
| Lepidolite | Honesty, stability, hope, acceptance. Assists in |
| | change and transition. Facilitates astral travel. |
| Malachite | Transformation, spiritual development. Clears the |
| | way to attain goals. Fidelity, loyalty, reasoning |
| | ability. |
| Moldavite | Clarity, eternity. Extra-terrestrial, interdimensional |
| | access. |
| Moonstone | Lunar, female energy. Emotional, intuitive. |
| | Rhythms, cycles, destiny. |
| Obsidian | Dispels negativity. Grounding, healing, protective. |
| | Helps one to clearly see one's flaws and the changes |
| | that are necessary. |
| Onyx | Centering, self-control, intuitive guidance. Assists in |
| | the grieving process. |
| Opal | Creativity, inspiration, imagination. |
| Peridot | Healing, protective. Allows one to understand |
| | changes in one's life, regulates life cycles. |
| Petrified Wood | Grounding, provides strength. Stone of |
| | transformation. |
| Pyrite | Shields from negative energy, good stone of |
| | protection. Enhances intellect and memory. Symbol |
| | of the sun. |
| Quartz Crystal | Universal crystal, clarity of consciousness. |
| (Clear) | |
| Rhodochrosite | Love energy. Excellent stone for balance in all areas. |
| Ruby | Loving, nurturing, spirituality, wealth, protection. |
| Rutilated Quartz | Intensifies energy of the crystal. Stimulates brain. |
| | Inspiration, clairvoyance. |
| Sapphire | Joy, peace, beauty, prosperity. |
| Selenite (Grpsum) | Clarity of consciousness, awareness, insight, good |
| | judgement. Aids in accessing past/future lives. |
| Silver | Mirror to the soul. Eloquence. Connects physical and |
| | astral bodies. In jewelry, it provides a balanced |
| | setting that retains the qualities of the stone it holds. |
| | Lunar energy. Cleansing, balancing. |
| Smoky Quartz | Dissolves negativity, grounding, balancing. Excellent |
| | for meditation. |
| Sodalite | Logic, efficiency, truthfulness. Enhances group |
| | communication. |
| Sugilite | Spiritual love, perfection, inspiration, confidence. |
| | Alleviates negative/destructive emotions. |
| Tigereye | Earthy, grounding. Represents sun and earth. |
| | Optimism, insight, personal power. |
| Topaz | Success, true love, individuality, creativity, joy. |
| Tourmaline | Inspiration, understanding, self-confidence, |
| | balancing. Electrical. |
| Tourmalinated | Strength, balancing. Combines attributes of |
| Quartz | tourmaline and quartz. |
| Turquoise | Spiritual attunement, strength, grounding. Protective - |
| | excellent for astral travel. |
| Zircon | Virtue, unification, continuity, purity. |

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

What is claimed is:

1. A gemstone infused product, the product comprising:
a container containing a fluid selected from perfume oil, fragrant water, nail polish or lip gloss;
a plurality of gemstones or combination of gemstones and simulants are placed inside the transparent container and covered by the fluid;
wherein the gemstones and simulants are not pulverized, emulsified or powdered;
wherein the plurality of gemstones or combination of gemstones and simulants are selected from a list consisting of Amethyst, Apatite, Beryl (Aquamarine, Bivite, Emerald, Goshenite, Heiodor, Morganite), Calcedony/Agate, Chrysoberyl, Citrine, Corundum, Diamond, Diopside, Feldspar, Fluorite, Garnet, Glass Tekites, Hematite, Hemimorphite, Iolite, Jade, Kyanite, Lapis Lazuli, Malachite, Opal, Peridot, Prehite, Quarts, Rhodochrisite, Scapolite, Sillimanite, Sphene, Spinel, Spodumene, Sugilite, Topaz, Tourmaline, Tugtupite, Turquoise, Zircon, and Zoisite, Yttrium Aluminium Garnet (YAG), Gadolinium gallium garnet (GGG), Cubic Zirconia and Glass Zircon.

2. The product of claim 1, further comprises a candle or a flower floating on the fluid.

3. The product of claim 4, wherein one or more of the plurality of gemstones or combination of gemstones and simulants have faceted cut that is Round, Brilliant, Briolette, Mixed, Fancy, Step, Square, Cross, French, Emerald, Asscher, Barrell, Antique, Ball, Needle, Old Mine, Rose, Star, Pear, Heart, Oval, Out of Round, Pentagon, Olive, Ceylon, Magna, Petal, Tycoon, Octagon, Triangle, Baguette, Radiant, Princess or combination thereof.

4. The product of claim 1, wherein the plurality of gemstones or combination of gemstones and simulants are reused after the fluid depletes.

5. A gemstone infused product, the product comprising:
a light color or transparent container containing clear lip gloss, perfume oil or nail polish;
a cap enclosing the container;
a plurality of gemstones or combination of gemstones and simulants are placed inside the transparent container and covered by the clear lip gloss, perfume oil or nail polish;
wherein the gemstones and simulants are not pulverized, emulsified or powdered;
wherein the plurality of gemstones or combination of gemstones and simulants are selected from a list consisting of Amethyst, Apatite, Beryl (Aquamarine, Bivite, Emerald, Goshenite, Heiodor, Morganite), Calcedony/Agate, Chrysoberyl, Citrine, Corundum, Diamond, Diopside, Feldspar, Fluorite, Garnet, Glass Tekites, Hematite, Hemimorphite, Iolite, Jade, Kyanite, Lapis Lazuli, Malachite, Opal, Peridot, Prehite, Quarts, Rhodochrisite, Scapolite, Sillimanite, Sphene, Spinel, Spodumene, Sugilite, Topaz, Tourmaline, Tugtupite, Turquoise, Zircon, and Zoisite, Yttrium Aluminium Garnet (YAG), Gadolinium gallium garnet (GGG), Cubic Zirconia and Glass Zircon.

6. The product of claim 5, wherein the plurality of gemstones or combination of gemstones and simulants are Peridot, Citrine and Cubic Zirconia.

7. The product of claim 5, wherein the plurality of gemstones or combination of gemstones and simulants are Rose Quartz, Garnet and Cubic Zirconic.

8. The product of claim 5, wherein the plurality of gemstones or combination of gemstones and simulants are Amethyst, Blue Topaz, Peridot, Citrine, Garnet and Cubic Zirconia.

9. The product of claim 5, wherein the clear lip gloss includes colored and transparent tint.

10. The product of claim 5, wherein the plurality of gemstones or combination of gemstones and simulants have faceted cut.

11. The product of claim 10, wherein the faceted cut is Round, Brilliant, Briolette, Mixed, Fancy, Step, Square, Cross, French, Emerald, Asscher, Barrell, Antique, Ball, Needle, Old Mine, Rose, Star, Pear, Heart, Oval, Out of Round, Pentagon, Olive, Ceylon, Magna, Petal, Tycoon, Octagon, Triangle, Baguette, Radiant, Princess or combination thereof.

12. A gemstone infused product, the product comprising:
a transparent container containing transparent perfume or oil, lip gloss or nail polish;
a plurality of gemstones or combination of gemstones and simulants are placed inside the transparent container and covered by the transparent perfume or oil, lip gloss or nail polish;
wherein the gemstones and simulants are not pulverized, emulsified or powdered;
wherein the plurality of gemstones or combination of gemstones and simulants are selected from a list consisting of Amethyst, Apatite, Beryl (Aquamarine, Bivite, Emerald, Goshenite, Heiodor, Morganite), Calcedony/Agate, Chrysoberyl, Citrine, Corundum, Diamond, Diopside, Feldspar, Fluorite, Garnet, Glass Tekites, Hematite, Hemimorphite, Iolite, Jade, Kyanite, Lapis Lazuli, Malachite, Opal, Peridot, Prehite, Quarts, Rhodochrisite, Scapolite, Sillimanite, Sphene, Spinel, Spodumene, Sugilite, Topaz, Tourmaline, Tugtupite, Turquoise, Zircon, and Zoisite, Yttrium Aluminium Garnet (YAG), Gadolinium gallium garnet (GGG), Cubic Zirconia and Glass Zircon.

13. The product of claim 12, wherein the plurality of gemstones or combination of gemstones and simulants are Amethyst, Blue Topaz and Cubic Zirconia.

14. The product of claim 12, wherein the plurality of gemstones or combination of gemstones and simulants are Rose Quartz, Garnet and Cubic Zirconia.

15. The product of claim 12, wherein the plurality of gemstones or combination of gemstones and simulants are Amethyst, Blue Topaz, Peridot, Citrine, Garnet and Cubic Zirconia.

16. The product of claim 12 further comprises a candle or a flower floating on the transparent perfume oil or fragrant liquid.

17. The product of claim 12, wherein the plurality of gemstones or combination of gemstones and simulants have faceted cut selected from Round, Brilliant, Briolette, Mixed, Fancy, Step, Square, Cross, French, Emerald, Asscher, Barrell, Antique, Ball, Needle, Old Mine, Rose, Star, Pear, Heart, Oval, Out of Round, Pentagon, Olive, Ceylon, Magna, Petal, Tycoon, Octagon, Triangle, Baguette, Radiant and Princess.
